(19) United States
(12) Patent Application Publication
(10) Pub. No.: US 2014/0127813 A1
(43) Pub. Date: May 8, 2014

(54) CYTOPLASMA TO ORGANELLE DELIVERY SYSTEM AND ASSOCIATED METHODS

(71) Applicant: Brigham Young University, Provo, UT (US)

(72) Inventors: Quentin T. Aten, Orem, UT (US); Regis A. David, Provo, UT (US); Sandra H. Burnett, Saratoga Springs, UT (US); Brian D. Jensen, Orem, UT (US); Larry L. Howell, Orem, UT (US)

(73) Assignee: Brigham Young University, Provo, UT (US)

(21) Appl. No.: 14/058,025

(22) Filed: Oct. 18, 2013

Related U.S. Application Data
(63) Continuation of application No. 13/681,260, filed on Nov. 19, 2012, now abandoned.
(60) Provisional application No. 61/561,159, filed on Nov. 17, 2011.

(51) Int. Cl. C12N 15/877

(52) U.S. Cl. CPC C12N 15/8775
USPC 435/461; 435/173.6; 435/283.1; 435/285.2

(57) ABSTRACT

Systems, devices, and methods for delivering a biological material into an organelle of a cell are provided. In one aspect, for example, a method for introducing biological material into an organelle of a cell can include bringing into proximity outside of a cell a lance and a preselected biological material, charging the lance with a polarity and a charge sufficient to electrically associate the preselected biological material with a tip portion of the lance, and penetrating an outer portion of the cell with the lance and directing and inserting the lance into the cell but outside of the organelle. The method can further include discharging the lance to release at least a portion of the biological material, charging the lance with an opposite polarity and charge sufficient to electrophoretically drive at least a portion of the biological material into the organelle, and withdrawing the lance from the cell.

### PRIORITY DATA

This application is a continuation of U.S. patent application Ser. No. 13/681,260, filed on Nov. 19, 2012, which claims the benefit of U.S. Provisional Patent Application Ser. No. 61/561,159, filed on Nov. 17, 2011, both of which are incorporated herein by reference.

### BACKGROUND OF THE INVENTION

Microinjection of foreign materials into a biological structure such as a living cell can be problematic. Various transfection techniques include the microinjection of foreign genetic material such as DNA into the nucleus of a cell to facilitate the expression of foreign DNA. For example, when a fertilized oocyte (egg) is transfected, cells arising from that oocyte will carry the foreign genetic material. Thus in one application, organisms can be produced that exhibit additional, enhanced, or repressed genetic traits. In some cases, researchers have used microinjections to create strains of mice that carry a foreign genetic construct causing macrophages to auto-fluoresce and undergo cell death when exposed to a certain drugs. Such transgenic mice have since played roles in investigations of macrophage activity during immune responses and macrophage activity during tumor growth.

Prior art microinjectors function in a similar manner to macro-scale syringes: a pressure differential forces a liquid through a needle and into the cell. In some cases a glass needle that has been fire drawn from a capillary tube can be used to pierce the cellular and nuclear membranes of an oocyte. Precise pumps then cause the expulsion of minute amounts of genetic material from the needle and into the cell. Researchers have produced fine microinjection needles made from silicon nitride and silica glass that are smaller than fire drawn capillaries. These finer needles generally also employ macro-scale pumps similar to those used in traditional microinjectors.

Pronuclear microinjection of DNA, for example, traditionally includes injection of liquid containing the DNA into the pronucleus of a cell such as a zygote. Such injections can be challenging processes due to the potential for cell lysis and chromosomal damage. In part, these challenges have motivated the development of various direct and indirect methods of transgenesis, such as viral transfection and embryonic stem cell targeting and injection. In viral transfection, a transgene is inserted into virus particles, which in turn act as carriers, delivering the genetic material to an oocyte or embryo. In embryonic stem-cell mediated transgenesis, a transgene is first targeted in vitro using a ubiquitous gene, such as ROSA, into embryonic stem cells. The transfected embryonic stem cells are then injected into blastocyst stage embryos, resulting in chimeric offspring. These chimeras must be bred to finally obtain germ line transgenic animals. In another example, existing micro-machined or carbon nanotube microelectromechanical systems (MEMS) designed for DNA delivery into tissue cultures have successfully introduced transgenes into cells, but are unsuitable for use in embryos for transgenic animal production. For example, such techniques require cells to grow around stationary needles or require extended periods of time to release bound DNA into the cells. Furthermore, such MEMS techniques do not provide sufficient mechanical displacement to penetrate a zygote's pronucleus.

### SUMMARY OF THE INVENTION

The present disclosure provides systems, devices, and methods for delivering a biological material into an organelle of a cell. In one aspect, for example, a method for introducing biological material into an organelle of a cell can include bringing into proximity outside of a cell a lance and a preselected biological material, charging the lance with a polarity and a charge sufficient to electrically associate the preselected biological material with a tip portion of the lance, and penetrating an outer portion of the cell with the lance and directing and inserting the lance into the cell but outside of the organelle. The method can further include discharging the lance to release at least a portion of the biological material, charging the lance with an opposite polarity and charge sufficient to electrophoretically drive at least a portion of the biological material away from the lance toward the organelle, and withdrawing the lance from the cell. In one specific aspect, charging the lance with the opposite polarity and charge is sufficient to electroporate the organelle's membrane. In one aspect, charging the lance with an opposite polarity and charge is sufficient to electrophoretically move at least a portion of the biological material from the lance and into the organelle.

In another aspect, the lance can be formed of a lance material that does not generate products toxic to the cell when the lance is charged. Additionally, the lance material can be selected to remain conductive under charged conditions. Furthermore, in some aspects the method can include bringing a counter electrode into electrical proximity of the lance to complete an electrical circuit. In one aspect, the counter electrode can be formed of an electrode material that does not generate products toxic to the cell when the lance is charged, and the electrode material remains conductive under charged conditions.

Non-limiting examples of organelles can include a nucleus, a pronucleus, a mitochondria, a chloroplast, a vacuole, an endocytic vesicle, a lysosome, and the like. In one specific aspect, the organelle can be a pronucleus. In another specific aspect, the biological material can be simultaneously delivered into two pronuclei of the same cell.

In another aspect, a method for transfecting a zygote with a biological material is provided. Such a method can include bringing into proximity a lance and a preselected DNA material outside of a zygote, charging the lance with a polarity and a charge sufficient to electrically associate the preselected DNA material with a tip portion of the lance, and penetrating an outer portion of the zygote with the lance and directing and inserting the lance into the cell but outside of the pronucleus. The method can further include discharging the lance to release at least a portion of the DNA material from the lance, charging the lance with an opposite polarity and charge sufficient to electrophoretically drive at least a portion of the DNA material into the pronucleus, and withdrawing the lance from the zygote.

In a further aspect, a system for electrophoretically introducing biological material into an organelle of interest of a cell is provided. Such a system can include a lance capable of receiving and holding an electrical charge sufficient to electrostatically associate preselected biological material thereto, a charging system electrically coupleable to the lance and operable to charge and discharge the lance, and a lance manipulation system operable to move the lance into and out of the cell. The charging system can be capable of delivering an electrical charge to the lance having a voltage in excess of a decomposition voltage of the lance that is sufficient to electrophoretically transport the preselected biological material into the organelle of interest. In one specific aspect, the charging system is capable of delivering a discontinuous voltage to the lance. In another specific aspect, the charging system includes a signal generator functionally coupled to a power supply such that the signal generator gates an electrical output of the power supply to generate the discontinuous voltage. In a further specific aspect, the charging system is capable of delivering both a positive and a negative electrical charge to the lance having a voltage in excess of the decomposition voltage of the lance. In yet a further aspect, the lance has a structural configuration to allow a portion of the lance to enter the cell and be positioned in sufficient proximity to the organelle of interest to effectively delivery the preselected biological material into the organelle of interest via electrophoresis. In another specific aspect, the lance has a structural configuration to allow a portion of the lance to enter the cell and be positioned in sufficient proximity to the organelle of interest that the organelle of interest is within an electroporetic envelope of the lance when the lance is charged.

### BRIEF DESCRIPTION OF THE DRAWINGS

FIG. 1A shows a schematic representation of a step of the delivery of a biological material into a cellular organelle in accordance with one embodiment of the present disclosure.

FIG. 1B shows a schematic representation of a step of the delivery of a biological material into a cellular organelle in accordance with one embodiment of the present disclosure.

FIG. 1C shows a schematic representation of a step of the delivery of a biological material into a cellular organelle in accordance with one embodiment of the present disclosure.

FIG. 1D shows a schematic representation of a step of the delivery of a biological material into a cellular organelle in accordance with one embodiment of the present disclosure.

FIG. 1E shows a schematic representation of a step of the delivery of a biological material into a cellular organelle in accordance with one embodiment of the present disclosure.

FIG. 1F shows a schematic representation of a step of the delivery of a biological material into a cellular organelle in accordance with one embodiment of the present disclosure.

FIG. 2A depicts a fragment of DNA in an electric field in accordance with another embodiment of the present disclosure.

FIG. 2B depicts a fragment of DNA in an electric field in accordance with another embodiment of the present disclosure.

FIG. 3 shows a simulation of an electric field around a lance in accordance with another embodiment of the present disclosure.

FIG. 4A shows a simulation of DNA movement in an electric field in accordance with another embodiment of the present disclosure.

FIG. 4B shows a simulation of DNA movement in an electric field in accordance with another embodiment of the present disclosure.

FIG. 5 shows a representation of pronuclear location in a cell in accordance with another embodiment of the present disclosure.

FIG. 6 shows a schematic of an injection system in accordance with another embodiment of the present disclosure.

FIG. 7 shows a schematic of an injection system in accordance with another embodiment of the present invention.

FIG. 8 shows a schematic of an injection system in accordance with another embodiment of the present invention.

FIG. 9A shows graphical representations of data in accordance with another embodiment of the present invention.

FIG. 9B shows graphical representations of data in accordance with another embodiment of the present invention.

FIG. 9C shows graphical representations of data in accordance with another embodiment of the present invention.

### DEFINITIONS OF TERMS

In describing and claiming the present invention, the following terminology will be used in accordance with the definitions set forth below.

The singular forms “a,” “an,” and, “the” include plural referents unless the context clearly dictates otherwise. Thus, for example, reference to “a support” can include reference to one or more of such supports, and reference to “an oocyte” can include reference to one or more of such oocytes.

As used herein, the term “biological material” can refer to any material that has a biological use and can be delivered into a cell or a cell organelle. As such, “biological material” can refer to materials that may or may not have a biological origin. Thus, such material can include natural and synthetic materials, as well as chemical compounds, dyes, and the like.

As used herein, the term “charging” can refer to the process of increasing the electrical charge on a structure such as a lance, regardless of the polarity of the charge. Thus, charging a lance can include increasing either the positive or negative electrical charge on the lance.

As used herein, the term “discharging” can refer to the process of decreasing the electrical charge on a structure such as a lance, regardless of the polarity of the charge. Thus, discharging a lance can include decreasing either the positive or negative electrical charge on the lance.

As used herein, the term “charged biological material” may be used to refer to any biological material that is capable of being attracted to or associated with an electrically charged structure. Accordingly, the term charged biological material may be used to refer to those molecules having a net charge, as well as those molecules that have a net neutral charge but possess a charge distribution that allows attraction to the structure.

As used herein, the term “uncharged” when used in reference to a lance may be used to refer to the relative level of charge in the lance as compared to a charged biological material. In other words, a lance may be considered to be “uncharged” as long as the amount of charge on the needle structure is insufficient to associate therewith a useable portion of the charged biological material. Naturally, what is a useable portion may vary depending on the intended use of the biological material, and it should be understood that one of ordinary skill in the art would be aware of what a useable portion is given such an intended use. Additionally it should be noted that a lance with no measurable charge would be considered “uncharged” according to the present definition.

As used herein, the term “associate” is used in one aspect to describe biological material that is in electrostatic contact with a structure due to attraction of opposite charges. For example, DNA that has been attracted to a structure by a positive charge is said to be associated or electrically associated with the structure.

As used herein, the term “sample” when used in reference to a sample of a biological material may be used to refer to a portion of biological material that has been purposefully attracted to or associated with the lance. For example, a sample of a biological material such as DNA that is described as being associated with a lance would include DNA that has been purposefully attracted thereto, but would not include DNA that is attracted thereto through the mere exposure of the lance to the environment. One example of DNA that would not be considered to be a “sample” includes airborne DNA fragments that may associate with the lance following exposure to the air.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. For example, a composition that is “substantially free of” particles would either completely lack particles, or so nearly completely lack particles that the effect would be the same as if it completely lacked particles. In other words, a composition that is “substantially free of” an ingredient or element may still actually contain such item as long as there is no measurable effect thereof.

As used herein, the term “about” is used to provide flexibility to a numerical range endpoint by providing that a given value may be “a little above” or “a little below” the endpoint without affecting the desired result.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 2, 3, 4, and 5, individually.

This same principle applies to ranges reciting only one numerical value as a minimum or a maximum. Furthermore, such an interpretation should apply regardless of the breadth of the range or the characteristics being described.

### DETAILED DESCRIPTION

Methods and associated systems for delivering biological material into an organelle within a cell are provided. As has been described, the delivery of a biological material into many cellular organelles can be challenging. The present disclosure describes a method capable of delivering a biological material into an organelle of a cell without physically penetrating the organelle with the delivery device. For convenience, this process can be termed Intracellular Electroporetic Nanoinjection (IEN).

Nanoinjection utilizes an injection structure such as a lance having at least a portion that is electrically conductive to hold a biological material with electrostatic charge during the delivery procedure. Using the presently disclosed techniques can facilitate the delivery of biological material into an organelle with enhanced results. As one non-limiting example, DNA can be delivered into the pronucleus of a zygote without physically penetrating the organelle with the lance, resulting in genomic integration of the DNA with increased embryo survival rates and increased progeny. While not intending to be bound to any scientific theory, such increased survival rates may be the result of reduced cellular damage from the DNA delivery.

Generally, the present methods and systems utilize the electrical association and dissociation of a biological material to a lance or other delivery device as a mechanism for delivering the biological material into a cell. Electroporation of intracellular structures from within the cell and subsequent electrophoretic movement of the biological material can effectively deliver the biological material into a cellular organelle with high rates of survival for the cell. In the case of embryonic cells, the increased survival of cells leads to higher survival rates of developing embryos and increased birth rates, leading to greater success in the survival to adulthood. Because the biological material can be loaded onto the lance and subsequently released via changes in the charge state of the lance, internal microinjection channels are not required for the delivery of the biological material into a cell or cellular organelle. As such, a lance can be smaller in size and can be formed in configurations that may not be possible with prior delivery devices. These delivery devices can have an outer shape and cross-section that is significantly smaller than traditional injection pipettes.

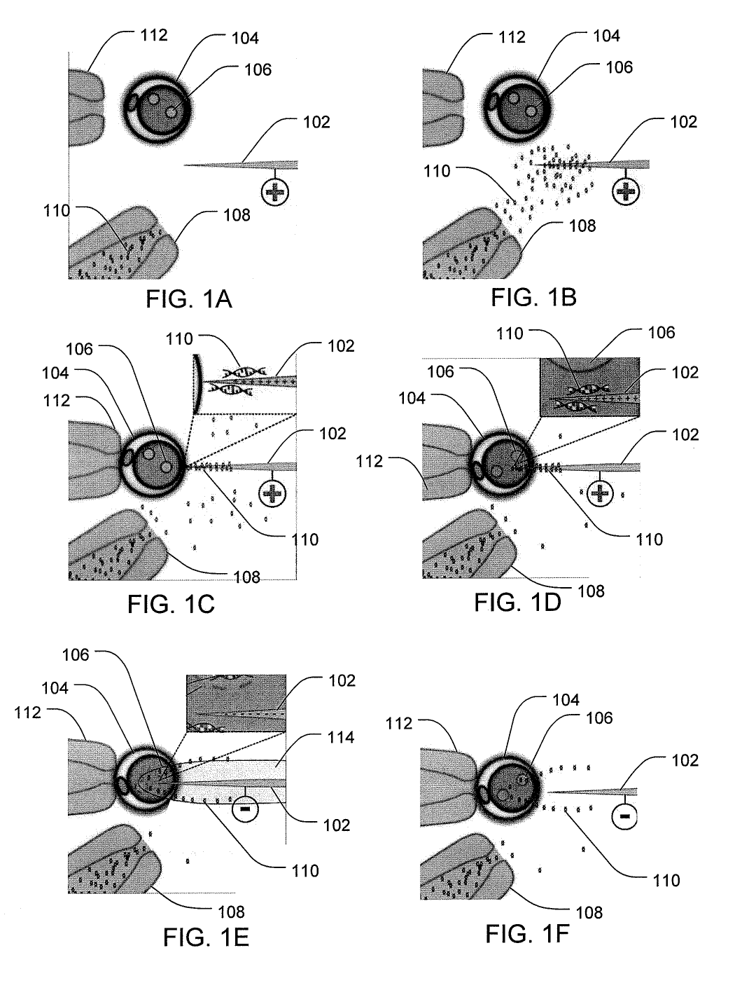

FIGS. 1A-F shows, for example, exemplary sequences of steps that can be performed to introduce biological material into an organelle of a cell. For this particular example, DNA is used as the biological material, a zygote is used as the cell, and a pronucleus is used as the organelle. This example is intended to be non-limiting, and the description can generally be applied to other biological materials, cells, organelles, and the like. Electrically-mediated delivery of DNA into an organelle can be accomplished due to the unequal charge distributions within DNA molecules. With an effective charge of 2 electrons per base pair, DNA can be manipulated by an electric field. FIG. 1A shows a lance 102 in proximity to a zygote 104 having a pronucleus 106. A biological material delivery device 108 (in this case a DNA placement micropipette) containing the biological material 110 (e.g. DNA in this case), is positioned in proximity to the tip portion of the lance 102. The biological material delivery device 108 is shown as a micropipette, however any device capable of delivery a biological material to the tip portion of the lance is considered to be within the present scope. A cell manipulation device 112 (i.e. a holding micropipette) is shown positioned in proximity to the zygote 104 to allow manipulation and/or securing of the cell during a biological material delivery procedure.

The lance 102 is charged with a polarity and a charge sufficient to electrically associate the biological material with a tip portion of the lance 102. In this case, the lance 102 is positively charged in preparation for the accumulation of DNA at a tip portion of the lance. A return or counter electrode is placed in electrical contact with the medium surrounding the lance in order to complete an electrical circuit with the charging device and the lance (not shown). The lance 102 is thus charged to a degree that is sufficient to associate DNA 110 to the lance during the injection procedure (FIG. 1B). The amount of voltage sufficient to charge the lance can vary depending on a variety of factors, such as the desired speed of the loading of DNA on the lance, the composition of the lance material, the electrochemical nature of the medium surrounding the lance, and the like.

Once the biological material has been electrically associated with a tip portion of the lance, the lance 102 can be inserted through an outer portion of the zygote 104 and into the cell's interior, as is shown in FIGS. 1C-D. It is noted that the lance tip can be inserted into any region of the cell allowing access to an organelle. In one aspect, for example, the lance can be inserted into the cytoplasm of a cell. In another aspect, the lance can be inserted into the nucleus of a cell. It is noted, however, that the lance does not penetrate the targeted organelle into which biological material delivery is to occur. With a tip portion of the lance 102 located within the zygote 104 (FIG. 1D), the lance 102 is discharged to release the biological material 110. Either subsequent to or concurrently with the release of the biological material, an oppositely charged current is delivered to the zygote 104 to create an electrical field 114 in order to electroporate the membrane of the organelle 106 (FIG. 1E). The biological material 110 is electrophoretically driven away from the lance tip and toward the organelle 106 by the electrical field 114. In some aspects, the lance 102 can be charged with an opposite polarity and charge that is sufficient to electrophoretically move at least a portion of the biological material 110 from the lance 102 and into the organelle 106 (FIGS. 1E-F). Electroporating in this manner allows delivery of a biological material into an organelle without the necessity of physical entry into the organelle by the lance. Eliminating the need to target the pronucleus for physical entry can increase the speed and ease of cellular injections. Additionally, cytoplasm-to-pronucleus nanoinjection can be insensitive to the size, location, and visibility of the organelle. This would be particularly beneficial in the generation of transgenic animals whose zygotes are difficult to transfect because of the visibility of their pronuclei.

It is noted that the above outlined procedures can be carried out with a single conductive lance or with multiple conductive lances or lance-like structures. For example, the biological material can be delivered with one lance, and the electroporation and electrophoresis can be provided by one or more additional lances or conductive elements. Additionally, in some aspects electroporation can occur simultaneously with electrophoresis.

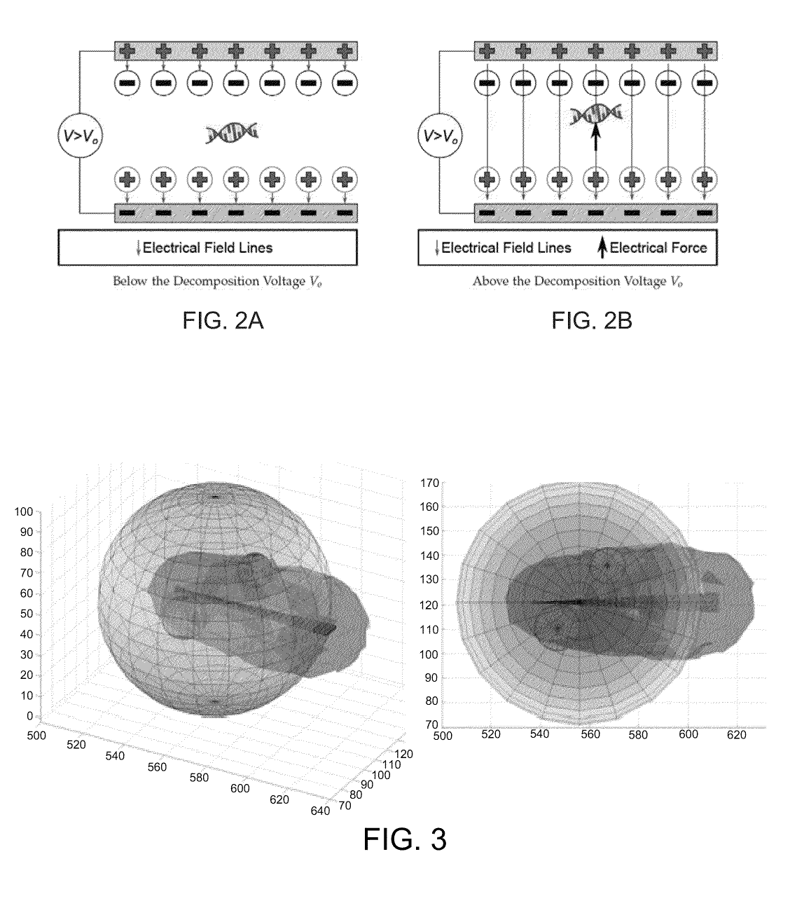

Electrophoretic motion, or the electric-field-driven motion of charged particles in an ionic solution, occurs when the voltage applied to the system exceeds a threshold called the decomposition voltage. Electrophoresis can be used to move molecules having a net charge, or an effective net charge like DNA, across micro-scale and macro-scale distances. Without intending to be bound to any scientific theory, the decomposition voltage is a consequence of the dissolved ions surrounding each electrode, called the electric double layer (EDL). As shown in FIG. 2A, below the decomposition voltage the EDL effectively shields the charges on the electrodes. Thus, a charged particle between the electrodes does not experience any electromotive force, and there is no electrical current flow due to the movements of ions in the solution. During microscale gel electrophoresis of DNA, with applied voltages below the decomposition voltage, no significant motion of the DNA is detected. Above the decomposition voltage, the electric field extends beyond the EDL and current flows between the electrodes through the movement of ions in the electrolyte solution. This principle is shown schematically in FIG. 2B, and is observed, for example, in the directed motion of DNA between electrodes during gel electrophoresis. The decomposition voltage depends upon such factors as the electrode material(s), the presence of a surface oxide on the electrode material, and the electrolyte solution's composition.

Electroporation is the process of opening micropores in the cell and/or organelle membranes by applying an electric field. Under normal conditions the membrane lipid bilayer surrounding the cell or nucleus is semipermeable, with the membrane lipid bilayer being impermeable to large and/or hydrophilic molecules. When a sufficiently large electric field is applied for microseconds to milliseconds, pores open in the membrane lipid bilayer within microseconds, and additional pores may also open through the denaturing of membrane proteins. These pores allow large and/or hydrophilic molecules such as DNA, RNA, or drugs to enter the cell, and simultaneously allow ions and macromolecules to leak from the cell into the surrounding media.

If the magnitude and duration of the pulses are small enough, the membrane pores will close in milliseconds to seconds, resulting in reversible electroporation. However, if the magnitude and duration of the pulses are too large, the membrane may undergo irreversible electroporation in some cases, in which the membrane lipid bilayer or protein pores close in minute to hours. Irreversible electroporation can result in cell lysis and cell death.

In one aspect, it can be beneficial to determine the metrics of the electroporation envelope. Careful design of the electroporation pattern can be useful in the prevention of cellular damage as a result of excessive electric field strength or duration. The electroporation envelope can be calculated as the region of the cell in which the electric field from the lance is greater than or equal to that required to open membrane pores. Simulations of various voltages can be run using a simulation models to find a voltage whereby the electroporation envelope around the lance would include the organelle being targeted; in one aspect, for example, both pronuclei can be targeted by the same electroporation envelope. While a variety of voltage ranges can be utilized depending on the particular details of a given injection procedure, in one aspect an appropriate repulsion voltage of 2 V above the decomposition voltage can be used. The decomposition voltage is empirically determined for each system and represents the voltage at which electrolysis begins. FIG. 3 shows a simulation output for the size of the electroporation envelope relative to a cell and pronuclei when 2 V above decomposition is applied to the lance. Large portions of the membranes of both pronuclei overlap with the electroporation envelope. These regions of the pronuclear membranes should experience transient pore formation giving transgene access to their interior. FIG. 3 shows that the electroporation envelope (blue) is the area of the cell expected to experience 200V/cm or greater when 2 volts above decomposition is applied to the lance. The electroporation envelope partially overlaps with both the male pronucleus (green) and female pronucleus (orange) causing pores to form in the nuclear membranes and allowing transgene to enter.

Voltages used for electroporation can include any voltage capable of electroporating a cellular organelle. In one aspect, for example, the electroporation voltage can be from about 0.25V to about 10V above the decomposition voltage. In another aspect, the electroporation voltage can be from about 0.25V to about 5V above the decomposition voltage. In yet another aspect, the electroporation voltage can be from about 0.25V to about 1V above the decomposition voltage. In a further aspect, the electroporation voltage can be from about 1V to about 5V above the decomposition voltage.

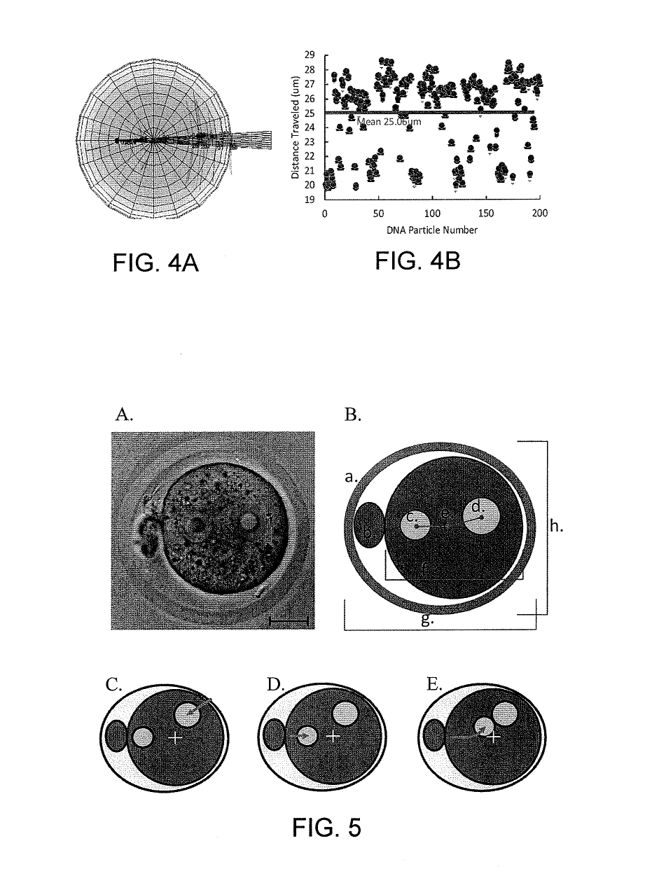

The voltages used to produce the electroporation envelope also induce electric field-driven motion of the biological material away from the lance. Although the biological material moves quickly, it does not reach a pronucleus instantaneously. The repulsion time thus should be sufficient for the biological material to travel through the cytoplasm and enter a pronucleus or other organelle. Computer modeling simulating a 1599 base pair transgene molecule being repelled at 2 volts above decomposition for various durations can identify suitable repulsion times. Ultimately the repulsion duration of 5 milliseconds was selected that produced an average repulsion distance of 25.06 μm (FIGS. 4A,B). A transgene molecule with the correct trajectory would thus reach the pronucleus and have a chance to enter through transient pores. FIG. 4A shows a computer simulation predicting the movement of multiple transgene molecules being repelled from a lance. Only a fraction of transgene molecule would have the correct trajectory to encounter a pronucleus. Points on the graph in FIG. 4B were generated results of 5 simulations each with 40 transgene molecules repelled for 5 milliseconds at 2 volts above decomposition (5.35V). The average repulsion distance obtained was 25.06 μm.

The electroporation current can be delivered as a continuous current or as a pulsed current. For example, a series of electrical pulses can induce a localized electroporation envelope around an organelle such as the pronuclei. The voltages are sufficient to induce electrophoretic motion of the biological material toward the organelle and produce pores in a biological membrane. In the case of the pronucleus, the lance can be positioned in the cytoplasm or the interior of the nucleus, and associated biological material can be electrophoretically driven into the pronucleus.

In some cases biological material can be electrophoretically driven into an organelle from the lance in a manner that minimizes deleterious effects to the cell. For example, delivering current from the lance above the decomposition voltage for extended periods can cause cellular damage. It can thus be beneficial to deliver electrophoretic or elecroporative current from the lance in a manner that reduces such negative impact on a cell. In one aspect, for example, charging the lance with an opposite polarity and charge further to move the biological material therefrom can include charging the lance with a time variant voltage signal. Thus the biological material moves in a time variant manner according to the time variant voltage signal. Such a time variant scheme allows biological material to move toward the organelle while at the same time reducing the exposure of the cell to current above the decomposition voltage per unit time. In other words, if it takes, for example, a biological material 10 seconds at 5V to move from the lance to the organelle, 5V delivered at a 50% duty cycle over 20 seconds would achieve the same result while reducing the cellular exposure per second to the 5V signal by half.

Various time variant voltage signals are contemplated, and any temporal voltage pattern is considered to be within the present scope. In one aspect, the time variant voltage signal can be a series of voltage or current pulses. Pulses can be of any shape, duration, duty cycle, or other temporal pattern. For example, pulses can be square waves, sine waves, triangle waves, aperiodic waves, or any other useful waveform. Similarly, pulse durations can include any pulse duration useful for electroporation or electrophoretic movement. In one aspect, pulse durations can be in a range for from about 1 microsecond to about 10 seconds. In another aspect, pulse durations can be in a range of from about 100 microseconds to about 5 seconds. In yet another aspect, pulse durations can be in a range of from about 0.5 ms to about 2 seconds. In a further aspect, pulse durations can be in a range of from about 10 nanoseconds to about 500 microseconds. In yet a further aspect, pulse durations can be in a range of from about 100 nanoseconds to about 75 microseconds.

A series of pulses can also be delivered at various duty cycles that can allow movement of the biological material while providing some protection to the cell. For example, in one aspect the duty cycle can be from about 1% to about 75%. In another aspect the duty cycle can be from about 10% to about 50%. Furthermore, in addition to delivering pulses at a fixed duty cycle, pulses can be delivered in an irregular timing pattern. Such a pattern can include fixed duration pulses delivered in a nonuniform timing pattern, variable duration pulses delivered in a uniform timing pattern, or variable pulse durations delivered in a nonuniform timing pattern. Additionally, the peak voltage distribution of the series of pulses can be constant or it can vary over time.

It is noted that the various pulse parameters described can be customized to optimize electrophoretic motion of the biological material while reducing potential damage to the cell. As such, a user can select pulse parameters to create a sequence of pulses that are effective for a particular cell, organelle, or experimental design.

Additionally, it is noted that biological material can be electrophoretically attracted to the lance during the initial charging phase outside of the cell in a similar manner to what has been described. As such, the lance can be charged with a voltage that is above the decomposition voltage to cause the active attraction of the biological material to the lance. Electrophoretic attraction can be accomplished with a steady current or a time variant voltage signal as has been described.

It can be beneficial to determine the approximate location of organelles such as pronuclei to maximize electroporation efficiency. For example, it is known that fertilization of a mouse oocyte initiates the migration of the male and female pronuclei toward each other to form a single metaphase plate for the first cell division, but the exact location of the pronuclei has not been established. In order to design a protocol able to produce an appropriately sized area of effect, it can be helpful to determine the location of pronuclei within the embryo during the time period injections would occur. As one example, an observational study was performed on healthy fertilized CD1 embryos to determine general embryo architecture including pronuclear size and location between 21-28 hours post hCG treatment (FIGS. 5A-E). Over 3000 images were taken using confocal microscopy and examined using digital analytical tools. General architecture measurements are presented in Table 1. The female pronucleus, defined as the smaller pronucleus and closest to the polar body, was found to have a biphasic migration pattern. Between 21-23 hours it is observed that the female pronucleus migrates toward the center of the embryo at an average rate of 2.8 μm/hr (P-value=0.029). During this same time span, the distance between male and female pronuclei does not change significantly (P-value=0.097). After 23 hours, the female pronucleus' migration rate towards the center of the embryo slows to 0.9 μm/hr (P-value=0.039). After 23 hours the distance between pronuclei begins to decrease (P-value=0.047), suggesting that the direction of migration of the female pronucleus shifts from the center of the embryo toward the male pronucleus around 23 hours (FIG. 4). No statistically significant change was detected in the location of the male pronucleus relative to the center of the embryo between 21-28 hours post hCG injections (P-value=0.228 for 21-23 hrs, and P-value=0.503 for 23-28 hrs), suggesting that the male pronucleus completes its migration toward the center prior to 21 hours. The center of the male pronucleus remains 15.95+/−5.46 μm from the center of the embryo between 21-28 hours. On average the center of the female pronucleus is located 21.07+/−4.51 μm from the center of the embryo. Based on these results, we concluded that the area of effect of the localized electroporation should extend at least 21 μm from the embryo center to ensure good coverage of the pronuclei throughout the time period in which IEN is performed. It is noted that the measurements described are for healthy fertilized CD1 embryos, and as such may vary for embryos of other strains and/or species.

TABLE 1

| | Size μm |
|---|---|
| Zona Length | 98.3 +/− 5.1 |
| Zona Width | 94.6 +/− 4.4 |
| Cell Diameter | 69.5 +/− 2.7 |
| Male Pronucleus Diameter | 19.3 +/− 1.7 |
| Female Pronucleus | 15.1 +/− 1.3 |
| Diameter | |

By locating an organelle such as a pronucleus in a cell, the lance can be positioned to more effectively deliver biological material thereto, even in cases where the organelle cannot be visualized, or is difficult to visualize.

It is noted that a variety of biological materials are contemplated for delivery into a cell or cellular organelle. The biological material can be a macromolecule or other material that exists outside of the cell that has been preselected for delivery into the cell. Various types of biological materials are contemplated for delivery into a cellular organelle, and any type of biological material that can be electrostatically delivered is considered to be within the present scope. Non-limiting examples of such biological materials can include DNA, cDNA, RNA, siRNA, tRNA, mRNA, microRNA, peptides, synthetic compounds, polymers, dyes, chemical compounds, organic molecules, inorganic molecules, and the like, including combinations thereof. In one aspect, the biological material can include DNA, cDNA, RNA, siRNA, tRNA, mRNA, microRNA, and combinations thereof. In another aspect, the biological material can include DNA and/or cDNA.

Biological material can be delivered to a variety of organelles, and any organelle capable of being targeted and receiving such biological material is considered to be within the present scope. Non-limiting examples of such organelles include nuclei, pronuclei, mitochondria, chloroplasts, vacuoles, endocytic vesicles, lysosomes, and the like. In one specific aspect, the organelle is a pronucleus. Similarly, both prokaryotic and eukaryotic cells are contemplated that can receive biological material, including cells derived from, without limitation, mammals, plants, insects, fish, birds, yeast, fungus, and the like. Additionally, cells can include somatic cells or germ line cells such as, for example, oocytes and zygotes. The enhanced survivability of cells with the present techniques can allow the use of cells and cell types that have previously been difficult to microinject due to their delicate nature.

The various types of organelles contemplated can vary significantly in size, and as such, delivery techniques used to introduce a biological material therein can be varied to accommodate the organelle. For example, organelles such as pronuclei, nuclei, chloroplasts, and vacuoles can be visualized using current optical microscopy. In these cases, a visual determination of the lance tip relative to the organelle can be used to orient the lance toward the organelle.

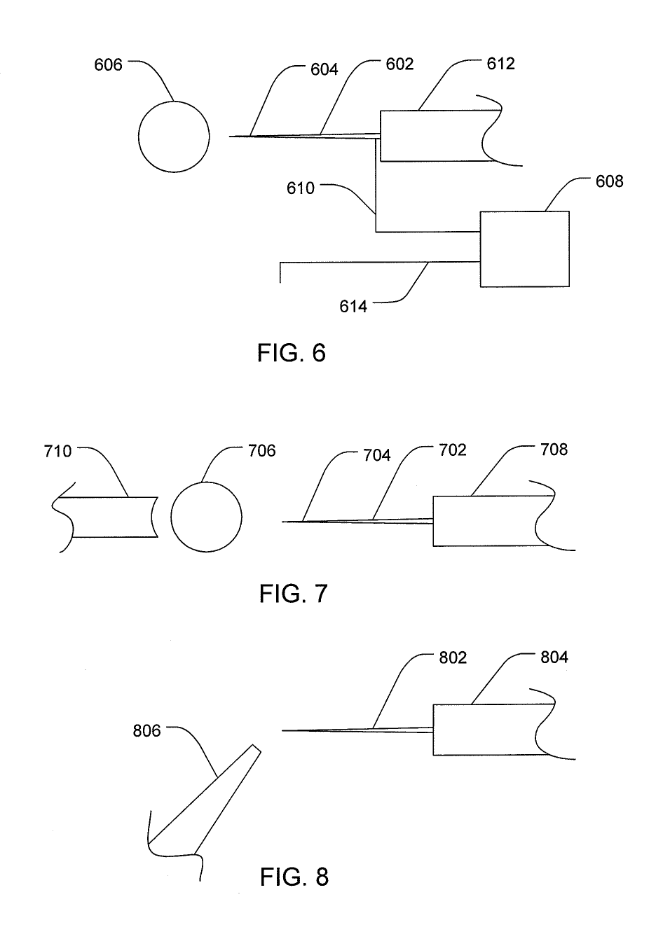

A variety of systems, system configurations, and system components are contemplated for use in delivering a biological material into an organelle of a cell, and any combination of components or configurations is considered to be within the present scope. As is shown in FIG. 6, for example, in one aspect a system for introducing biological material into an organelle of a cell can include a lance 602 having a working portion 604 operable to enter a cell 606, where the working portion has a maximum diameter selected to effectively deliver biological material to an organelle while minimizing damage to the cell. The system can also include a charging system 608 electrically coupleable 610 to the lance 602 and being operable to charge and discharge the lance 602, and a lance manipulation system 612 operable to move the lance 602 into and out of a cell in a reciprocating motion along an elongate axis of the lance that minimizes damage to the cell 606. The system can also include a return 614 for completing an electrical circuit with the charging system 608.

In another aspect, as is shown in FIG. 7, a system for introducing biological material into an organelle of a cell can include a lance 602 having a working portion 604 operable to enter a cell 606. The system can also include a lance manipulation system 608 operable to move the lance 602 into and out of an organelle in a reciprocating motion along an elongate axis of the lance that minimizes damage to the cell 606. The system can also include a cell manipulation device 610 for holding the cell 606 during a biological material delivery procedure.

In yet another aspect, as is shown in FIG. 8, a system for introducing biological material into an organelle of a cell can include a lance 802 and a lance manipulation system 804 operable to move the lance 802 into and out of an organelle in a reciprocating motion along an elongate axis of the lance that minimizes damage to the cell. The system can also include a biological material delivery device 806 configured to deliver a biological material capable of association with the lance 802. As has been described, the biological material delivery device 806 can be positioned to release biological material in the proximity of a tip portion of the lance 802.

As has been described, a lance can be configured to be inserted into a cell in a position to electrophoretically deliver a biological material into an organelle. As such, the physical configuration of such a lance should be sufficient to allow penetration into the cell while minimizing damage to cellular structures. In one aspect, for example, the lance can be a narrow tapered structure having a tip diameter capable of penetrating the organelle while minimizing damage. The physical configuration of the lance can, in some cases, vary depending on the desired configuration of electrical field. Thus, the physical configuration of a given lance can be designed according to the type of cell, the type of organelle, and/or the organelle location within the cell.

Accordingly, any size and/or shape of lance capable of delivering biological material into an organelle is considered to be within the present scope. The size and shape of the lance can also vary depending on the organelle receiving the biological material. The effective diameter of the lance, for example, can be sized to improve the survivability of the cell. It should be noted that the term “diameter” is used loosely, as in some cases the cross section of the lance may not be circular. Limits on the minimum effective diameter of the lance can, in some cases, be a factor of the material from which the lance is made and the manufacturing process used. In one aspect, for example, the lance can have a tip diameter of from about 5 nm to about 3 microns. In another aspect, the lance can have a tip diameter of from about 10 nm to about 2 microns. In another aspect, the lance can have a tip diameter of from about 30 nm to about 1 micron. In a further aspect, the lance can have a tip diameter that is less than or equal to 1 micron. As such, in many cases the tip diameter of the lance can be smaller than the resolving power of current optical microscopes, which is approximately 1 micron. As is noted above, lance tips are contemplated that can have cross sections that are not circular. In such cases, it is intended that the circumference of a circle defined by the tip diameters disclosed above would be substantially the same as an outer circumferential measurement of a non-circular lance tip. One non-limiting example of a non-circular lance tip can have a thickness of about 0.5 to about 2.0 microns and a width of about 17 to about 200 nanometers.

The length of the lance can be variable depending on the design and desired attachment of the lance to the lance manipulation system. Also, the portion of the lance that is contacting and/or passing through a portion of the cell can vary in length depending on the lance design and the depth of the organelle into which the biological material is to be delivered. For example, delivering biological material to an organelle located near the surface of a cell can be accomplished using a shorter lance as compared to delivery to an organelle located deep within the cell. This would not preclude, however, the use of longer lances for delivery into organelles near the cellular surface. For example, a relatively long lance may be used to deliver biological material in an application where only a small portion (e.g., only the tip) of the lance penetrates a cell. It should be noted that the lance length can be tailored to the delivery situation and to the preference of the individual performing the delivery.

Thus the length of the lance can be any length useful for a given delivery operation. For example, in some aspects, the lance can be up to many centimeters in length. In other aspects, the lance can be from a millimeter to a centimeter in length. In another aspect, the lance can be from a micron to a millimeter in length. In one specific aspect, the lance can be from about 2 microns to about 500 microns in length. In another specific aspect, the lance can be from about 2 microns to about 200 microns in length. In yet another specific aspect, the lance can be from about 10 microns to about 75 microns in length. In a further specific aspect, the lance can be from about 40 microns to about 60 microns in length.

Additionally, the shape of the lance, at least through the portion of the lance contacting the cell, can vary depending on the design of the lance and the depth to which the biological material is to be injected into the cell. A high lance taper, for example, may be more disruptive to cellular membranes and internal cellular structures than a low taper. In one aspect, for example, the lance can have a taper of from about 1% to about 10%. In another aspect, the taper can be from about 2% to about 6%. In yet another aspect, the taper can be about 3%. The taper of the lance can also be described in terms of the size of the disruption in the cell membrane following insertion. In one aspect, for example, the approximate diameter of the disrupted area of the cell membrane following lance insertion is from about 10 nanometers to about 8 microns. In another aspect, the approximate diameter of the disrupted area of the cell membrane following lance insertion is from about 2 micron to about 5 microns.

The overall shape and size of the lance can also be designed to take into account various factors, including those involved with the delivery procedure, as well as the materials utilized to make the lance. For example, in one aspect a lance can be designed having sufficient cross sectional strength to allow biological material delivery, while at the same time minimizing the damage done to the cell from the lance's cross sectional area. As another example, the lance can be designed to have a cross sectional area sufficient to minimize damage to the cell, while at the same having sufficient surface area to which biological material can be electrically associated.

Different materials can also affect the design of the size and shape of the lance. Some materials may not hold a charge sufficient to associate the biological material to the lance tip at smaller sizes. In such cases, larger size lances can be used to facilitate a higher charge capacity. It may be difficult in some cases to form particular sizes and shapes of the lance from certain materials. In such cases, the lance size and shape can be designed to the properties of the desired material. For example, a material such as gold may not be capable of supporting the lance tip at very small diameters due to inadequate strength at smaller sizes, or it may not be possible or feasible to create a very small diameter tip with gold. If the use of a gold lance is desired, the lance size and shape can thus be designed with the properties of gold in mind.

As has been described, a charge can be introduced into and held by the lance in order to electrically associate the biological material to the lance. Various lance materials are contemplated for use in constructing the lance, and any material that can be formed into a lance structure and is capable of carrying a charge is considered to be within the present scope. Non-limiting examples of lance materials can include a metal or metal alloy, a conductive glass, a polymeric material, a semiconductor material, and the like, including combinations thereof. Non-limiting examples of metals can include indium, gold, platinum, silver, copper, palladium, tungsten, aluminum, titanium, and the like, including alloys and combinations thereof. Polymeric materials that can be used to construct the needle structure can include any conductive polymer, non-limiting examples of which include polypyrrole doped with dodecyl benzene sulfonate ions, SU-8 polymer with embedded metallic particles, and the like, including combinations thereof. Non-limiting examples of useful semiconductor materials can include germanium, gallium arsenide, and silicon, including various forms of silicon such as amorphous silicon, monocrystalline silicon, polycrystalline silicon, and the like, including combinations thereof. Indium-tin oxide is a material that is also contemplated for use as a lance material. Furthermore, in one aspect the lance can be substantially solid.

Additionally, in some aspects the lance can be a conductive material that is coated on a second material, where the second material provides the physical structure of the lance. Examples can include metal-coated glass or metal-coated quartz lances. The lance can also include a hollow, non-conductive material, such as a glass, where the hollow material is filled with a conductive material. Depending on the design, the lance can be manufactured using various techniques such as wire pulling, chemical etching, MEMs processing, various deposition techniques, and the like.

Additionally, in some aspects the material utilized to construct the lance and/or the counter electrode can be a material that does not generate products toxic to the cell when the lance is charged. For example, a material such as chrome can form chrome oxides upon electrolysis that are toxic to many cells. Choosing a material (e.g. gold, tungsten, platinum, iridium, stainless steel) that does not generate such toxic compounds can reduce potential damage to the cell. Additionally, a material can be utilized that remains conductive under charged conditions. For example, a material that oxidizes under charging conditions may be problematic if such oxidization lowers the conductivity of the lance or counter electrode. It is also noted that a material that is useful when the lance is functioning as an anode may not be a useful material for the same lance when functioning as a cathode.

The charging system can include any system capable of electrically charging, maintaining the charge, and subsequently discharging the lance. Non-limiting examples can include batteries, DC power supplies, photovoltaic cells, static electricity generators, capacitors, signal generators, digital-to-analog converters, direct digital synthesis integrated circuits, and other charging systems capable of producing steady state or time variant voltages. The charging system can include a switch for activation and deactivation, and in some aspects can also include a polarity switch to reverse polarity of the charge on the lance. In one aspect the system may additionally include multiple charging systems, one system for charging the lance with a charge, and another charging system for charging the lance with an opposite polarity charge. In one exemplary scenario, an initially uncharged lance is brought into contact with a sample of a biological material. The biological material can be in water, saline, or any other liquid capable of maintaining biological material. A charge opposite in polarity to the biological material is applied to the lance, thus associating a portion of the biological material with the lance. The lance can then be moved into the organelle of interest, and lance can be discharged, thus releasing the biological material.

The lance can be manipulated by any system or mechanism capable of aligning and moving the lance. Non-limiting examples of lance manipulation systems include mechanical systems, magnetic systems, piezoelectric systems, electrostatic systems, thermo-mechanical systems, pneumatic systems, hydraulic systems, and the like. In one aspect, the lance manipulation system can be one or more micromanipulators. The lance may also be moved manually by a user. For example, a user may push the lance along a track from first location to a second location.

In one aspect, the lance can be moved by the lance manipulation system in a reciprocal motion along an elongate axis of the lance. In other words, the lance can move forward into a cell and backward out of the cell along the same path. By moving along the elongate axis of the lance, the minimum cross sectional area of the lance is driven through cellular structures such as a cell membrane. This minimal cross sectional exposure can limit the cellular disruption, and thus potentially increasing the success of the biological material delivery procedure.

Further exemplary details regarding lances, charging systems, lance manipulation systems, and cellular restraining systems can be found in U.S. patent application Ser. No. 12/668,369, filed Sep. 2, 2010; Ser. No. 12/816,183, filed Jun. 15, 2010; and 61/380,612, filed Sep. 7, 2010, each of which is incorporated herein by reference.

It should be noted that the design of a system for delivering biological material into an organelle of a cell can vary due to the interdependencies of various system parameters. Combinations of features can thus influence other features, both in terms of system design and in terms of system use. Features can thus be mixed and matched to create a delivery system for a given purpose or desirable performance. For example, the materials and configuration chosen for the lance may have properties allowing a greater or lesser charge capacity, thus influencing the voltage, current, and electrical timing of the charging and discharging. A smaller tip diameter can more effectively enter an organelle with potentially less damage, but may have a smaller surface area for the association of biological material. The association capacity of the lance for biological material can thus be increased, for example, by utilizing lance materials capable of holding a higher relative charge, or by utilizing a non-circular shape for the lance tip that increases surface area while minimizing the penetration damage of the lance. Thus, if a particular feature is desired for a lance, other features can be varied to accommodate such a design. As such, it should be understood that the various details described herein should not be seen as limiting, particularly those involving dimensions or values. It is contemplated that a wide variety of design choices are possible, and each are considered to be within the present scope.

### EXAMPLES

The following examples are provided to promote a more clear understanding of certain embodiments of the present invention, and are in no way meant as a limitation thereon.

### Example 1

### Confocal Microscopy, Imaging, and Analysis of Embryos for Pronuclear Migration Study

Harvested CD1 mouse embryos were incubated in hyaluronidase, rinsed in M2 medium (Sigma cat #M7167), rinsed again in PBS, then cultured in KSOM medium and incubated at 37° C. and 5% CO² until the time of imaging. Embryos were transferred to a welled microscope slide, inverted, and placed into position on the microscope platform. Embryos that showed signs of polyspermy, fragmentation of the cytoplasm, or lack of fertilization were discarded. 106 quality embryos were imaged and used to perform the measurements to acquire the data presented. An Olympus Confocal Laser Scanning Biological Microscope FV300 and Fluoview software were used for image acquisition and analysis respectively. Embryos were imaged using the 60× objective.

Z-stacks were captured in 1.5 or 2 μm increments to produce 3D images. Measurements of the cell membrane (not the zona) were used to identify the widest portion of the embryo. The midpoints of diameter in both X and Y planes were used to identify the center of the embryo. All subsequent measurements were taken from that point. Basic geometry formulae were used to determine distances between items not in the same plane. Between 30-50 images were taken per cell to produce the present measurements. The data presented for general embryo architecture represent the mean+/−one standard deviation.

### Example 2

### Transgene Preparation

The pCX-GFP plasmid encodes enhanced green fluorescent protein (EGFP) under the control of the chicken β-actin promoter (CAG). Transgene was prepared for injection by restriction digest with Stu 1 and Spe 1, followed by gel electrophoresis and purification with QIAEX II kit (Qiagen, Valencia, Calif.). For Intracellular Electroporetic Nanoinjection (IEN), the transgene was diluted to 10-15 ng/μl in PBS. For microinjection, the transgene was diluted to a concentration of 2-3 ng/pi in low (0.1M) EDTA TE (pH 7.4).

### Example 3

### Mice, Zygotes, and Embryos

In vivo mouse work was performed at the University of Utah Transgenic and Gene Targeting Mouse Core in Salt Lake City, Utah. All animal use was in accordance with guidelines of the Animal Welfare Act and followed protocols approved by the Institutional Animal Care and Use Committee (IACUC). Female C57Bl/6J×CBA/J F1 mice were treated with 5 units pregnant mare serum gonadotropin (PMS) (NHPP, Torrance, Calif.) 3 hrs prior to the dark cycle, and were treated 47 hours later with 5 units of human chorionic gonadotropin (hCG) (Sigma cat #CG-10) and co-housed with stud males. Donor embryos were harvested 18 hours after hCG injection from females with a vaginal plug by dissection of cumulus mass from the oviducts. Cumulus mass was incubated in 800 units/ml of hyaluronidase (Sigma cat #H4272) in M2 medium (Sigma cat #M7167) for two minutes. Embryos were rinsed in M2 medium, then maintained in a drop of M16 medium (Sigma cat #M7292) under mineral oil (Sigma cat #M8410) at 37° C. and 5% CO₂.

For microinjection, zygotes with obvious pronuclei were chosen for injection. After IEN and microinjection zygotes were cultured in a 50 μl drop of M16 under oil overnight. Healthy two-cell stage embryos were rinsed three times in 100 μl drops of M2 and surgically implanted into the oviducts of 0.5 day pseudo-pregnant females. Timed pseudo-pregnant females were obtained by mating C57Bl/6J×CBA/J F1 females to vasectomized C57Bl/6J×CBA/J F1 males and checking for vaginal plugs. Approximately twenty two-cell embryos were implanted per mouse.

### Example 4

### MEMS Nanoinjection Devices

Nanoinjector design, fabrication and release were performed as described previously (Aten et al. (2012) Nanoinjection: pronuclear DNA delivery using a charged lance. Transgenic Res. doi:10.1007/s11248-012-9610-6, incorporated herein by reference). The release process was followed by thorough rinses in sterile deionized water and isopropanol. The chip was then in a clean and sterile condition. The released chip was adhesively bonded to the inner side of a 35 mm cell culture dish lid. The lid served as the dish for submerging the chip in phosphate buffered saline (PBS) during IEN.

### Example 5

### IEN and Microinjection

For the microinjection versus IEN comparative study, all harvested zygotes were pooled and made available to the microinjection technician and the nanoinjection technician. All transfer surgeries of two-cell stage embryos into surrogate females occurred the day following injections.

IEN was performed in PBS at room temperature. Each batch of zygotes remained on the MEMS chip for less than 30 minutes. The nanoinjection lance held (+)1.5 volts while 0.25 μl of ˜15 ng/ul DNA solution was dispensed over the lance from a holding pipette using a syringe pump. The (+) voltage was maintained to allow DNA accumulation for 30-90 seconds. The (+) voltage was also maintained during insertion of the lance into the embryo. The lance tip was positioned in the cytoplasm and the DNA was repelled using 10 0.5 millisecond pulses 2 volts above decomposition voltage for a total repelling time of 5 milliseconds. The lance was then withdrawn from the zygote. No attempt was made to locate or pierce the pronuclei during injections.

Microinjection was performed in M2 under oil at room temperature. Using a standard procedure, one or both pronuclei were located visually, and the zygote was repositioned appropriately to allow the microinjection needle to pierce the pronuclear membrane. Two picoliters of a 2-3 ng/μl DNA solution were microinjected using an Eppendorf Femtojet microinjection system until there was obvious slight swelling of the pronucleus.

### Example 6

### Genotypic and Phenotypic Testing

DNA was purified using Qiagen DNeasy tissue kit. Each sample was subjected to PCR for EGFP as well as for mouse β-actin using EGFP primers (forward 5′-ATGGTGAGCAAGGGCGAGGA-3′ (SEQ ID 001) and reverse 5′-TTGTACAGCTCGTCCATCCG-3′ (SEQ ID 002)) to yield a 716 bp product and mouse β-actin primers (forward 5′-GTGGGCCGCTCTAGGCACCA-3′ (SEQ ID 003) and reverse 5′-CGGTTGGCCTTAGGGTTCAGGG-3′ (SEQ ID 004)) to yield a 244 bp product. EGFP PCR products from representative mice were submitted to the Brigham Young University DNA Sequencing Center for Big Dye sequencing. DNA samples from PCR positive pups and WT controls were submitted to TransViragen (Research Triangle Park, NC) for southern blot analysis. For southern blotting, genomic DNA samples were digested with PstI and blots were hybridized with a 716 bp chemiluminescent probe (forward primer 5′-ATGGTGAGCAAGGGCGAGGA-3′ (SEQ ID 001), reverse primer 5′-TTGTACAGCTCGTCCATCCG-3′ (SEQ ID 002)).

Flow cytometry was performed on samples of thigh muscle, brain, spleen, and gut to detect EGFP expression. Samples were homogenized in 2 ml of Hanks, and passed through a 70 μm filter. Readings were obtained with a BD Biosciences FACSCanto cytometer and were analyzed using Diva software (BD Biosciences).

### Example 7

### Statistical Analysis of Survival, Integration, and Expression Data

The viability, birth, integration, and expression data between IEN and microinjection groups were compared using statistical methods for the two-tailed Fisher's exact test for 2×2 contingency tables using the statistical software package SAS (S.A.S Institute). Confidence intervals were produced using the Jeffrey's interval. The Jeffrey's confidence intervals were calculated with a confidence level of (1−α) for x successful events (births, expressing pups, etc.) out of n attempts (injections performed, pups observed, etc.) with α=0.05.

### Example 8

### Production of Transgenic Animals Via IEN

To demonstrate the effectiveness of IEN, a comparative study was performed using pronuclear microinjection as a positive control. CAG-EGFP transgene was injected into fertilized embryos using either pronuclear microinjection or IEN. Pronuclear microinjections were performed by a trained lab technician; IEN was performed by a technician trained in the use of IEN techniques. All other aspects of embryo harvest, culture, and implantation were identical between the two groups. Injections occurred over the course of two days and included over 600 injected embryos. Viability of injected embryos was recorded 24 hours after injection and at birth to compare survivability of the procedures. There was no statistical difference in the viability of eggs at 24 hours post-injection between IEN and microinjection (P=0.688). There was also no statistical difference between the injection methods in the number of pups born per transferred embryo (P=0.205).

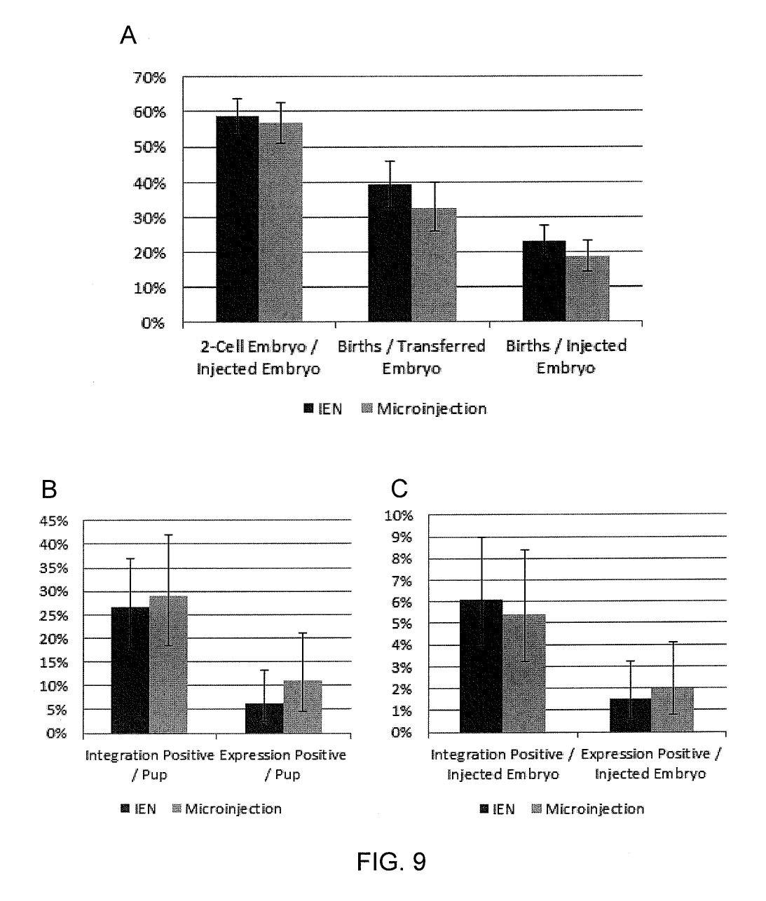

After birth and weaning, pups were tested for transgene integration and expression using PCR analysis and flow cytometery respectively. Sequencing of PCR product and southern blot analysis were performed on a subset of individuals to confirm integration was accurately detected. IEN successfully produced both integration and expression positive pups. Survival, integration, and expression data are shown in Table 2 and FIGS. 9A-C. There was no significant difference between IEN and microinjection in the integration of transgene when observing integration per pup (P=0.8447) and integration per injected zygote (P=0.7375). There was also no statistical difference when comparing the expression rate per injected zygote (P=0.7621). The Fisher's exact test confirmed that IEN and microinjection survival, integration, and expression rates are not significantly different; thus, we conclude that IEN is both safe and effective in comparison to microinjection.

TABLE 2
Survival, integration, and expression data for IEN and microinjection. There is
no statistical difference between IEN rates and microinjection rates according
to Fisher's exact test at the 0.05 level.
A. Data on embryo viability, birth rate per viable embryo, and the number
of pups with positive integration and expression as observed in the
IEN-versus-microinjection comparison study.

| | 2-cell Embryos/ | Births/ | | |
|---|---|---|---|---|
| | Injected | Transferred | Integration | Expression |
| | Embryo | Embryo | Positive/Pup | Positive/Pup |
| IEN | 202/344 | 79/202 (39.1%) | 21/79 (26.5%) | 5/79 (6.3%) |
| | (58.7%) | | | |
| Microinjection | 168/295 | 55/168 (32.7%) | 16/55 (29.1%) | 6/55 (10.9%) |
| | (56.9%) | | | |

B. Data from the IEN-versus-microinjection comparison study reported
per injected zygote.

| | | | Integration | Expression |
|---|---|---|---|---|
| | 2-cell Embryos/ | | Positive/ | Positive/ |
| | Injected | Births/ | Injected | Injected |
| | Embryo | Injected Embryo | Embryo | Embryo |
| IEN | 202/344 | 79/344 (23.0%) | 21/344 (6.1%) | 5/344 (1.5%) |
| | (58.7%) | | | |
| Microinjection | 168/295 | 55/295 (18.6%) | 16/295 (5.4%) | 6/295 (2.0%) |
| | (56.9%) | | | |

FIG. 9 shows IEN and microinjection survival, integration, and expression rates. A) Embryo survival rates and birth rates for IEN and microinjection were not statistically different. B) Integration and expression rates per pup were not statistically different between groups. C) Integration and expression rates per injection were not statistically different between groups. Plotted confidence intervals are Jeffery's 95% confidence intervals for binomial proportions.

The general architecture measurements for CD 1 mouse embryos produced are comparable to other published reports. Although cell diameter measurements were somewhat shorter at 69.5+/−2.7 μm than some published diameters, these reports did not mention measuring tools and may represent estimates only. It appears that the pronuclear migration model presented is the first documentation of the biphasic migration pattern of the female pronucleus in mice. The biphasic migration may prove to be a common feature of mammalian embryo development. While CD1 embryos were used for measurements, the protocols developed based on the measurement results were successfully tested on embryos derived from C57Bl/6J×CBA/J F1 females. It is anticipated that the present protocol could be applied to most mouse strains and likely to other organisms as well.

The results demonstrate that it is possible to target the location of the pronuclei within an embryo for transgene delivery without visualizing the pronucleus. A voltage of 2 volts above decomposition (for a total of approximately 5.35V) yields a 200V/cm electroporation envelope which encloses at least part of the membranes of both pronuclei without electroporating the entire embryo. A total voltage pulse duration of 5 milliseconds similarly provides the transgene molecules with sufficient opportunity to reach a pronucleus while pores are present in the pronuclear membranes. Survival data indicates that IEN is at least as viable as microinjection.

The results of the in vivo comparative study demonstrate that IEN is a safe and effective cytoplasmic delivery technology. Cytoplasmic delivery of transgene via microinjection was first attempted by Brinster in 1985, but did not result in transgenic animals. The transgene was either degraded in the cytoplasm before nuclear transport could occur or lacked signals necessary for transport altogether. Attempts have been made to stabilize foreign DNA by encapsulation in liposome and thereby enable integration, but have also failed to produce transgenic animals. One report described the use of poly-L-lysine added to transgene which allowed for transgene integration. The poly-L-lysine contribution to integration was unknown, but both increased stability and nuclear uptake are possible. It is posited that IEN is a successful delivery strategy because it facilitates nuclear uptake of the transgene and prevents prolonged exposure of the transgene to the cytoplasm thereby preventing enzymatic degradation. Similar rates of IEN and microinjection transgene integration and expression are not surprising as integration will proceed through non-homologous recombination regardless of the method of delivery to the pronucleus.

Cytoplasmic injections by IEN offer a number of advantages over pronuclear injections. Cytoplasmic injections are faster and easier because all injections can be performed identically into the center of the embryo without regard to the location of the pronuclei. Injection into the cell center rather than into a pronucleus eliminates the time-consuming positioning required for an ideal pronuclear injection. IEN may be particularly beneficial when working with embryos for which microinjection is already challenging. Some livestock such as pigs have embryos which are not transparent. These embryos must be centrifuged prior to injection to allow the pronuclei to be visualized. Additionally some strains of mice have smaller than average pronuclei that are difficult to visualize and inject, making microinjection challenging. IEN injections would be unaffected by either of these scenarios.

IEN may offer other intriguing possibilities which have not yet been pursued. First, electroporation of transgene into other cellular structures such as the mitochondria. Manipulation of the mitochondrial genes through transgenesis could be beneficial for studying metabolic functions and diseases. There is currently no established technique for specific transgenesis of mitochondrial genes in higher organisms. Second, the use of zinc finger nucleases in combination with IEN to produce transgenic animals which are homozygous for the desired transgene insertion. Since IEN and zinc fingers target both pronuclei a portion of transgenic animals produced in this manner could have two identical integration events. Overall IEN shows the potential to contribute a great deal to the fields of transgenic and biological research.

It is to be understood that the above-described compositions and modes of application are only illustrative of preferred embodiments of the present invention. Numerous modifications and alternative arrangements may be devised by those skilled in the art without departing from the spirit and scope of the present invention and the appended claims are intended to cover such modifications and arrangements. Thus, while the present invention has been described above with particularity and detail in connection with what is presently deemed to be the most practical and preferred embodiments of the invention, it will be apparent to those of ordinary skill in the art that numerous modifications, including, but not limited to, variations in size, materials, shape, form, function and manner of operation, assembly and use may be made without departing from the principles and concepts set forth herein.

1. A method for introducing biological material into an organelle of a cell, comprising:
bringing into proximity outside of a cell a lance and a preselected biological material;
charging the lance with a polarity and a charge sufficient to electrically associate the preselected biological material with a tip portion of the lance;
penetrating an outer portion of the cell with the lance and directing and inserting the lance into the cell but outside of the organelle;
discharging the lance to release at least a portion of the biological material;
charging the lance with an opposite polarity and charge sufficient to electrophoretically drive at least a portion of the biological material away from the lance toward the organelle; and
withdrawing the lance from the cell.

2. The method of claim 1, wherein charging the lance with the opposite polarity and charge is sufficient to electroporate organelle membrane.

3. The method of claim 1, wherein the organelle includes a member selected from the group consisting of a nucleus, a pronucleus, a mitochondria, a chloroplast, a vacuole, an endocytic vesicle, and a lysosome.

4. The method of claim 1, wherein the organelle is a pronucleus.

5. The method of claim 4, wherein the biological material is simultaneously delivered into two pronuclei of the same cell.

6. The method of claim 1, wherein inserting and withdrawing the lance is performed with a reciprocating motion along an elongate axis of the lance.

7. The method of claim 1, wherein the lance is formed of a lance material that does not generate products toxic to the cell when the lance is charged, and wherein the lance material remains conductive under charged conditions.

8. The method of claim 7, further includes bringing a counter electrode into electrical proximity of the lance to complete an electrical circuit, and wherein the counter electrode is formed of an electrode material that does not generate products toxic to the cell when the lance is charged, and wherein the electrode material remains conductive under charged conditions.

9. The method of claim 1, wherein the biological material includes a member selected from the group consisting of DNA, RNA, peptides, polymers, organic molecules, inorganic molecules, ions, and combinations thereof.

10. The method of claim 1, wherein the biological material includes DNA.

11. The method of claim 1, wherein charging the lance with an opposite polarity and charge is sufficient to electrophoretically move at least a portion of the biological material from the lance and into the organelle.

12. The method of claim 1, wherein charging the lance with an opposite polarity and charge further includes charging the lance with a time variant voltage signal such that the biological material moves in a time variant manner according to the time variant voltage signal.

13. The method of claim 12, wherein the time variant voltage signal is a series of voltage pulses.

14. The method of claim 13, wherein the series of voltage pulses have a duty cycle of from about 5% to about 50%.

15. The method of claim 13, wherein the series of voltage pulses have a pulse duration of from about 100 ns to about 75 microseconds.

16. The method of claim 13, wherein the series of voltage pulses has an irregular peak voltage distribution.

17. The method of claim 12, wherein the time variant voltage signal has an irregular timing pattern.

18. The method of claim 1, wherein charging the lance with a polarity and a charge sufficient to electrically associate the preselected biological material with a tip portion of the lance further includes charging the lance with a polarity and charge sufficient to electrophoretically attract and move the preselected biological material to the lance.

19. A method for transfecting a zygote with DNA, comprising:
bringing into proximity a lance and a preselected DNA material outside of a zygote;
charging the lance with a polarity and a charge sufficient to electrically associate the preselected DNA material with a tip portion of the lance;
penetrating an outer portion of the zygote with the lance and directing and inserting the lance into the zygote but outside of a pronucleus;
discharging the lance to release at least a portion of the DNA material from the lance;
charging the lance with an opposite polarity and charge sufficient to electrophoretically drive at least a portion of the preselected DNA material into the pronucleus; and
withdrawing the lance from the zygote.

20. The method of claim 19, wherein electrophoretically driving at least a portion of the preselected DNA material into the pronucleus further includes simultaneously delivering the preselected DNA material into two pronuclei of the zygote.

21. A system for electrophoretically introducing biological material into an organelle of interest of a cell, comprising:
a lance capable of receiving and holding an electrical charge sufficient to electrostatically associate preselected biological material thereto;
a charging system electrically coupleable to the lance and operable to charge and discharge the lance, the charging system being capable of delivering an electrical charge to the lance having a voltage in excess of a decomposition voltage of the lance that is sufficient to electrophoretically transport the preselected biological material into the organelle of interest; and
a lance manipulation system operable to move the lance into and out of the cell.

22. The system of claim 21, wherein the charging system is capable of delivering a discontinuous voltage to the lance.

23. The system of claim 22, wherein the charging system includes a signal generator functionally coupled to a power supply such that the signal generator gates an electrical output of the power supply to generate the discontinuous voltage.

24. The system of claim 21, wherein the charging system is capable of delivering both a positive and a negative electrical charge to the lance having a voltage in excess of the decomposition voltage of the lance.

25. The system of claim 21, wherein the lance manipulation system is operable to move the lance into and out of the cell in a reciprocating motion along an elongate axis of the lance that minimizes damage to the cell.

26. The system of claim 21, wherein the lance has a structural configuration to allow a portion of the lance to enter the cell and be positioned in sufficient proximity to the organelle of interest to effectively delivery the preselected biological material into the organelle of interest via electrophoresis.

27. The system of claim 21, wherein the lance has a structural configuration to allow a portion of the lance to enter the cell and be positioned in sufficient proximity to the organelle of interest that the organelle of interest is within an electroporetic envelope of the lance when the lance is charged.


SEQUENCE LISTING (4 sequences)

SEQ ID NO: 1 - DNA, 20 bp, mouse
atggtgagcaagggcgagga

SEQ ID NO: 2 - DNA, 20 bp, mouse
ttgtacagctcgtccatccg

SEQ ID NO: 3 - DNA, 20 bp, mouse
gtgggccgctctaggcacca

SEQ ID NO: 4 - DNA, 22 bp, mouse
cggttggccttagggttcaggg